(19) United States
(12) Patent Application Publication
(10) Pub. No.: US 2023/0042817 A1
(43) Pub. Date: Feb. 9, 2023

(54) ANALYTE CAPTURE FROM AN EMBEDDED BIOLOGICAL SAMPLE

(71) Applicant: 10x Genomics, Inc., Pleasanton, CA (US)

(72) Inventor: Marco Mignardi, Stockholm (SE)

(21) Appl. No.: 17/881,086

(22) Filed: Aug. 4, 2022

Related U.S. Application Data
(60) Provisional application No. 63/229,313, filed on Aug. 4, 2021.

(51) Int. Cl. C12Q 1/6841; C12N 15/10

(52) U.S. Cl. CPC C12Q 1/6841; C12N 15/1096; C12Q 1/6834

(57) ABSTRACT

Provided herein are methods, compositions, and kits, for capturing analytes from an embedded biological sample and spatially barcoding the analytes with an array.

### CROSS-REFERENCE TO RELATED APPLICATIONS

This application claims priority to U.S. Provisional Patent Application No. 63/229,313, filed on Aug. 4, 2021. The contents of that application are incorporated herein by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provides substantial analyte data for dissociated tissue (i.e., single cells), but fail to provide information regarding the position of the single cell in a parent biological sample (e.g., tissue sample).

Sample preparation for spatial assays, particularly for formalin-fixed paraffin-embedded (FFPE) samples, presents issues that affect sensitivity and spatial resolution: under-permeabilized tissue will not effectively release analytes (e.g., mRNA) while over-permeabilized samples can lose spatial resolution due to excessive diffusion of the analyte from its place of origin. Permeabilization conditions can affect analyte (e.g., mRNA) release and permeabilization conditions have to be optimized for different types of biological samples (e.g., different tissue sources). The methods disclosed herein overcome optimization steps by embedding the biological sample, cross-linking analytes within the embedded biological sample, and subsequently removing the non-crosslinked portions of the biological sample.

Cross-linked analytes, including those from different layers of the biological sample can be subsequently released from the embedded sample and captured on an array, thereby increasing efficiency. Additionally, loss of spatial resolution due to increased diffusion can be limited by different strategies (e.g., affinity binding, electrophoresis, or use of magnetic particles).

Analytes and/or cDNA primers can be captured on a first substrate to generate a cDNA array that contains the analytes' spatial and sequence information. The cDNA array can be probed (e.g., from above) with a spatially barcoded array. This method allows spatial barcodes to be added to the cDNA array and the nucleic acid sequence of the analytes to be added to the spatially barcoded array, generating two complementary barcoded slides (“replica plates”). Alternatively, primers on the spatially barcoded array can be blocked so that the same spatially barcoded array can be re-used (e.g., one or more times) to spatially probe multiple analytes or samples.

The methods disclosed herein address the following problems: (1) the need to optimize permeabilization conditions for different biological samples (e.g., fresh-frozen, fixed, or FFPE samples); (2) the need to optimize sensitivity and efficiency, for example, capturing more analytes from different layers of the biological sample; and (3) the need to place the biological sample precisely onto the array with minimal loss of spatial information.

### SUMMARY

To avoid having to optimize permeabilization conditions for different biological samples (e.g., fresh-frozen, fixed or FFPE samples) or different tissue types (e.g., breast, lung, tonsil, mouse brain), the methods described herein place a biological sample on a substrate including second binding moiety. In some examples, the biological sample is stained, imaged, and/or permeabilized. Hydrogel-binding probes can be delivered to the permeabilized biological sample, where analytes (e.g., nucleic acids) can bind (e.g., hybridize) to a portion (e.g., a poly(dT) sequence) of the hydrogel-binding probes. In some examples, the hydrogel-binding probes also contain hydrogel-binding moieties (e.g., acrydite). In some embodiments, the hydrogel-binding probes include a first binding moiety (e.g., a first binding moiety that interacts with a second binding moiety). In some examples, the biological sample can be contacted (e.g., perfused) with an embedding material (e.g., polyacrylamide, agarose, any other embedding materials described herein) such that the hydrogel-binding moieties (e.g., acrydite) are cross-linked to the embedding material. In some examples, after the analytes are captured by the hydrogel-binding probes cross-linked to the embedding material, the biological sample can be cleared. For example, proteins can be cleared from the biological sample by a proteinase (e.g., pepsin, Proteinase K, etc.) and peptides, lipids, other nucleic acids can be removed by washes. In some examples, cross-linked analytes (e.g., nucleic acids) can be released by disassembling the embedding material or disassembling the hydrogel-binding probes and letting the analytes (e.g., nucleic acids) diffuse to the substrate. Diffusion can be partially controlled by 1) electrophoresis; 2) labelling the hydrogel-binding probes with a first binding moiety (e.g., to bind a Proteinase K resistant second binding moiety-coated substrate); 3) with magnetic particles which are attracted to the substrate by a magnetic force; or 4) combinations thereof. In some examples, analytes (e.g., nucleic acids) are bound to the substrate via the first binding moiety and second binding moiety complex and primed to make cDNA. In some examples, the hydrogel-binding probes act as primers to generate cDNAs based on the sequence of the analytes. In some examples, the analytes (e.g., mRNA) are digested by a nuclease (e.g., RNaseH) leaving behind single-stranded cDNAs on the substrate. In some examples, the cDNAs are extended with a terminal transferase (e.g., TdT) and/or a poly(A) polymerase to add on one or more additional nucleotides. In some examples, the additional nucleotides are d(A) nucleotides. In some examples, poly(A) tails can be ligated to the single-stranded cDNAs. In some examples, the cDNAs can be captured by a spatially barcoded array containing spatially barcoded capture probes including a poly(dT) capture domain which hybridizes to the added poly(A) sequence (e.g., a capture sequence) on the single-stranded cDNAs. In some examples, for each pair of single-stranded cDNA and spatially barcoded capture probe, both strands (e.g., the capture probe and the cDNA) can be extended using the other strand as an extension template, thereby adding a spatial barcode to the single-stranded cDNA and a complement of the single-stranded cDNA sequence to the capture probe. In some examples, complementary cDNA libraries are generated.

In some examples, the spatially barcoded array contacted with the single-stranded cDNAs can contain capture probes where the 3′ end of the capture domain (e.g., poly(dT) sequence) is blocked. In some examples, the capture probes can be partially or completely blocked. In some examples, the blocking probe includes an overhang poly(dT) sequence that can hybridize to the added nucleotides (e.g., poly(dA)) on the single-stranded cDNA. In some examples, the single-stranded cDNA is primed and the spatial barcode, or a complement thereof, are added to the single-stranded cDNA. In some examples, the spatially barcoded array can be re-used to prime another array. In some examples, another array with different spatial barcodes can be used to prime the single-stranded cDNA on a “shifted position” (e.g., about 100 μm shift) to cover a larger area of the biological sample.

In some examples, anchoring of analytes (e.g., nucleic acids) to the embedding material (e.g., hydrogel) can be achieved in several ways. For example, non-limiting methods include hybridizing to the poly(A) tail of the analyte (e.g., mRNA) via a hydrogel-binding probe or by chemical modification of the analytes (e.g., RNA nucleotides) to covalently bind to the embedding material (e.g., acrylamide). In some examples, migrating analytes to the substrate can be accomplished by different means (e.g., via electrophoresis or magnetic field). In some examples, binding of the analyte to the substrate can be done using different binding moiety pairs (e.g., biotin and avidin, SpyTag and SpyCatcher).

Provided herein are methods for determining a location of an analyte in a biological sample, the method including: (a) contacting the biological sample with a plurality of hydrogel-binding probes, where a hydrogel-binding probe of the plurality of hydrogel-binding probes includes (i) a first capture domain, where the first capture domain binds to the analyte; (ii) a first binding moiety; and (iii) a hydrogel-binding moiety; (b) embedding the biological sample in a hydrogel including a plurality of hydrogel subunits, where the hydrogel-binding probe is crosslinked to a hydrogel subunit of the plurality of hydrogel subunits via the hydrogel-binding moiety; (c) releasing the analyte bound to the hydrogel-binding probe from the hydrogel, thereby allowing the analyte bound to the hydrogel-binding probe to bind to a substrate including a plurality of second binding moieties, where a second binding moiety of the plurality of second binding moieties binds to the first binding moiety; (d) extending a 3′ end of the hydrogel-binding probe to generate an extended probe using the analyte bound to the first capture domain as a template and adding a capture sequence to the 3′ end of the extended probe; (e) contacting the substrate including the extended probe with an array including a plurality of capture probes, where a capture probe includes (i) a second capture domain, where the second capture domain binds the capture sequence of the extended probe, and (ii) a spatial barcode; (f) extending the extended probe to generate a first strand using the capture probe as a template; and (g) determining the sequence of (i) the spatial barcode, or a complement thereof, and (ii) all or a portion of a sequence corresponding to the analyte, or a complement thereof, and using the sequences of (i) and (ii) to determine the location of the analyte in the biological sample.

In some embodiments, step (f) includes extending the capture probe on the array to generate a second strand using the extended probe as a template.

In some embodiments, the method includes before step (f), blocking the 3′ end of the extended capture probe on the array.

In some embodiments, the hydrogel-binding probe includes a cleavage domain, a unique molecular identifier, one or more functional domains, or any combination thereof.

In some embodiments, the analyte is RNA, and optionally, where the RNA is mRNA.

In some embodiments, the first capture domain of the hydrogel-binding probe is a poly(T) sequence, the second capture domain includes a poly(T) sequence, and the capture sequence includes a homopolymeric sequence, where the homopolymeric sequence is a poly(A) sequence.

In some embodiments, the hydrogel-binding moiety is located at a 5′ end of the hydrogel-binding probe and where the hydrogel-binding moiety includes acrydite.

In some embodiments, the method includes, after step (b), and before step (c) clearing the biological sample, where the clearing includes the use of one or more of a lipase, an RNase, a DNase, and a protease, where the protease includes Proteinase K and, optionally, one or more washing steps.

In some embodiments, the plurality of second binding moieties and the first binding moiety of the hydrogel-binding probe are resistant to degradation by proteinase K. In some embodiments, after step (d), removing the analyte from the hydrogel, where the removing includes the use of an RNase.

In some embodiments, the method includes, before step (a), a step of permeabilizing the biological sample and optionally before step (b) staining and imaging the biological sample.

In some embodiments, step (c) includes disassembling the hydrogel, where the hydrogel includes a reversible cross-linker. In some embodiments, step (c) includes actively migrating the analyte bound to the hydrogel-binding probe to the substrate, where the step of actively migrating the analyte bound to the hydrogel-binding probe to the substrate includes electrophoresis.

In some embodiments, the hydrogel-binding probe includes an agent that decreases migration of the analyte bound to the hydrogel-binding probe.

In some embodiments, step (c) includes removing the agent that decreases migration from the hydrogel-binding probe and after removing the agent that decreases migration from the hydrogel-binding probe, a step of actively migrating the analyte bound to the hydrogel-binding probe to the substrate via electrophoresis.

In some embodiments, the hydrogel-binding probe includes a magnetic particle and where step (c) includes actively migrating the analyte bound to the hydrogel-binding probe to the substrate using a magnetic field.

In some embodiments, the hydrogel-binding probe includes a charged particle and where step (c) includes actively migrating the analyte bound to the hydrogel-binding probe to the substrate using electrophoresis.

In some embodiments, the adding of the capture sequence to the 3′ end of the extended probe in step (d) includes the use of one or more enzyme(s), where the one or more enzymes is a DNA ligase, a terminal deoxynucleotidyl transferase, or a poly(A) polymerase.

In some embodiments, the extending of the 3′ end of the hydrogel-binding probe in step (d) is performed using a reverse transcriptase and the extending of the 3′ end of the extended probe in step (f) is performed using a DNA polymerase.

In some embodiments, the first binding moiety includes biotin and the second binding moiety includes streptavidin or the first binding moiety includes streptavidin and the second binding moiety includes biotin, the plurality of hydrogel subunits include acrylamide, and the hydrogel includes a polyacrylamide hydrogel.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIGS. 2A-2G shows an exemplary scheme of capturing analytes in a biological sample.

FIGS. 3A-31I shows an exemplary scheme of spatially barcoding analytes with a spatially barcoded array.

### DETAILED DESCRIPTION

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by (e.g., secreted) and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination, and each of which is incorporated herein by reference in their entireties. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain. In some embodiments, a capture probe can further include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

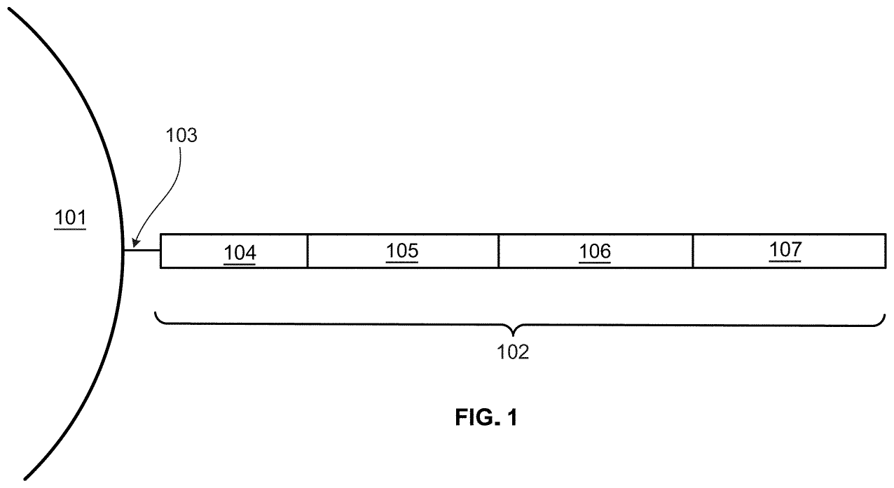

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature. In some embodiments, the UMI sequence 106 of a capture probe attached to a given feature is different from the UMI sequence of a different capture probe attached to the given feature.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication No. 2021/0140982A1, U.S. Patent Application No. 2021/0198741A1, and/or U.S. Patent Application No. 2021/0199660.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in WO 2021/102003 and/or U.S. patent application Ser. No. 16/951,854, each of which is incorporated herein by reference in their entireties.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2021/102039 and/or U.S. patent application Ser. No. 16/951,864, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, WO 2021/102005, and/or U.S. patent application Ser. No. 16/951,843, each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

Capturing Analytes from Embedded Biological Samples

The methods of this disclosure generally describe methods for determining the location of an analyte (e.g., nucleic acid) in a biological sample including the use of embedded biological samples. Provided herein are methods for determining a location of an analyte (e.g., mRNA) in a biological sample (e.g., a fresh frozen, fixed or FFPE sample) including contacting the biological sample with a plurality of hydrogel-binding probes, where a hydrogel-binding probe of the plurality of hydrogel-binding probes includes (i) a first capture domain (e.g., a poly(dT) sequence), where the first capture domain binds to the analyte; (ii) a first binding moiety (e.g., biotin); and (iii) a hydrogel-binding moiety (e.g., acrydite); embedding the biological sample in a hydrogel (e.g., polyacrylamide) including a plurality of hydrogel subunits, where the hydrogel-binding probe is crosslinked to a hydrogel subunit of the plurality of hydrogel subunits via the hydrogel-binding moiety; releasing the analyte bound to the hydrogel-binding probe from the hydrogel, thereby allowing the analyte bound to the hydrogel-binding probe to bind to a substrate including a plurality of second binding moieties (e.g., streptavidin), where a second binding moiety of the plurality of second binding moieties binds to the first binding moiety; extending a 3′ end of the hydrogel-binding probe to generate an extended probe (e.g., cDNA) using the analyte bound to the first capture domain as a template and adding a capture sequence (e.g., a poly(A) sequence) to the 3′ end of the extended probe; contacting the substrate comprising the extended probe with an array comprising a plurality of capture probes, where a capture probe includes (i) a second capture domain (e.g., a poly(dT) sequence), where the second capture domain binds the capture sequence of the extended probe, and (ii) a spatial barcode; extending the extended probe to generate a first strand using the capture probe as a template; and (g) determining the sequence of (i) the spatial barcode, or a complement thereof, and (ii) all or a portion of a sequence corresponding to the analyte, or a complement thereof, and using the sequences of (i) and (ii) to determine the location of the analyte in the biological sample.

Also provided herein are methods for determining the location of an analyte (e.g., mRNA) in a biological sample (e.g., a fresh frozen, fixed or FFPE tissue sample) including contacting the biological sample with a plurality of hydrogel-binding probes, where a hydrogel-binding probe of the plurality of hydrogel-binding probes includes (i) a first capture domain (e.g., a poly(dT) sequence), where the first capture domain binds to the analyte; (ii) a first binding moiety (e.g., biotin); and (iii) a hydrogel-binding moiety (e.g., acrydite); embedding the biological sample in a hydrogel (e.g., polyacrylamide) including a plurality of hydrogel subunits, where the hydrogel-binding probe is crosslinked to a hydrogel subunit of the plurality of hydrogel subunits via the hydrogel-binding moiety; releasing the analyte bound to the hydrogel-binding probe from the hydrogel, thereby allowing the analyte bound to the hydrogel-binding probe to bind to a substrate including a plurality of second binding moieties (e.g., streptavidin), where a second binding moiety of the plurality of second binding moieties binds to the first binding moiety; extending a 3′ end of the hydrogel-binding probe to generate an extended probe (e.g., cDNA) using the analyte bound to the first capture domain as a template and adding a capture sequence (e.g., a poly(dA) sequence) to the 3′ end of the extended probe; contacting the substrate comprising the extended probe with an array including a plurality of capture probes, where a capture probe includes (i) a second capture domain (e.g., a poly(dT) sequence), where the second capture domain binds the capture sequence of the extended probe, and (ii) a spatial barcode; extending the capture probe on the array to generate an extended capture probe using the extended probe as a template; and determining the sequence of (i) the spatial barcode, or a complement thereof, and (ii) all or a portion of a sequence corresponding to the analyte, or a complement thereof, and using the sequences of (i) and (ii) to determine the location of the analyte in the biological sample.

In some embodiments, step (f) includes extending the capture probe on the array to generate a second strand using the extended probe as a template.

In some embodiments, the biological sample is a tissue section. In some embodiments, the biological sample is a fixed biological sample. In some embodiments, the fixed biological sample is formalin-fixed paraffin-embedded (FFPE) biological sample. In some embodiments, the biological sample is placed on a substrate (e.g., a slide or die from a wafer). In some embodiments, the substrate is a streptavidin coated substrate. In some embodiments, the biological sample is stained (e.g., H&E or DAPI) and imaged. In some embodiments, the biological sample is permeabilized (e.g., permeabilized by any of the permeabilization methods described herein).

In some embodiments, the method for determining the location of an analyte in a biological sample includes contacting the biological sample with a plurality of hydrogel-binding probes, where a hydrogel-binding probe of the plurality of hydrogel-binding probes has (i) a first capture domain (e.g., a poly(dT) sequence), where the first capture domain binds to the analyte; (ii) a first binding moiety (e.g., biotin); and (iii) a hydrogel-binding moiety (e.g., acrydite). In some embodiments, the analyte is RNA. In some embodiments, the RNA is mRNA. In some embodiments, the capture domain of the hydrogel-binding probe is a poly(dT) sequence that binds (e.g., hybridizes) to mRNA. In some embodiments, the mRNA is captured by the hydrogel-binding probes. In some embodiments, the hydrogel-binding moiety is located at a 5′ end of the hydrogel-binding probe. In some embodiments, the hydrogel-binding moiety contains an acrydite moiety.

In some embodiments, the method for determining the location of an analyte in a biological sample includes embedding the biological sample in a hydrogel including a plurality of hydrogel subunits, where the hydrogel-binding probe is cross-linked to a hydrogel subunit of the plurality of hydrogel subunits via the hydrogel-binding moiety. In some embodiments, one or more of the hydrogel subunits include acrylamide. In some embodiments, the hydrogel is a polyacrylamide hydrogel. In some embodiments, the analyte is captured by a hydrogel subunit via the hydrogel-binding moiety of the hydrogel-binding probe. In some embodiments, the biological sample is embedded (e.g., perfused) by an acrylamide solution which forms a mesh, which binds to the acrydite moiety on the hydrogel-binding probe. In some embodiments, cross-linking an analyte (e.g., mRNA) to a hydrogel subunit is performed by chemical modification of the analyte to form a covalent bond with the hydrogel (e.g., acrylamide).

In some embodiments, the method for determining the location of an analyte in a biological sample further includes clearing the biological sample. In some embodiments, clearing the biological sample includes the use of one or more of a proteases, a lipase, an RNAse and a DNAse. In some embodiments, the biological sample is cleared by proteinase K. In some embodiments, the one or more second binding moieties on the substrate and the one or more first binding moieties on the hydrogel-binding probe are resistant to Proteinase K. In some embodiments, clearing the biological sample includes the use of one or more washes. In some embodiments, after Proteinase K treatment, peptides, lipids, and DNA are removed by one or more washes and only the cross-linked analytes are left in the acrylamide mesh on the substrate.

In some embodiments, the method for determining the location of an analyte in a biological sample further includes releasing the analyte bound to the hydrogel-binding probe from the hydrogel. In some embodiments, the analyte is released from the hydrogel by disassembling the hydrogel. In some embodiments, the hydrogel has a reversible cross-linker. In some embodiments, the analyte is released from the hydrogel by disassembling the hydrogel-binding probe. In some embodiments, the hydrogel-binding probe has a cleavage domain, a unique molecular identifier, one or more functional domains, or combinations thereof. In some embodiments, the analyte is released from the hydrogel by enzymatic cleavage of the hydrogel-binding probe.

In some embodiments, the method for determining the location of an analyte in a biological sample further includes allowing the analyte bound to the hydrogel-binding probe to bind to a substrate comprising a second binding moiety (e.g., streptavidin) that can bind to the first binding moiety (e.g., biotin). In some embodiments, the analyte y bound to the hydrogel-binding probe is migrated to the substrate by active migration. In some embodiments, the hydrogel-binding probe has an agent that decreases migration of the analyte bound to the hydrogel-binding probe. In some embodiments, the agent that decreases migration is removed from the hydrogel-binding probe before active migration. In some embodiments, the analyte is migrated to the substrate by electrophoresis. In some embodiments, the analyte bears a negative or positive charge. In some embodiments, the hydrogel-binding probe bears a negative or positive charge. In some embodiments, the hydrogel-binding probe includes a magnetic particle. In some embodiments, the analyte is migrated to the substrate by magnetic force. In some embodiments, the substrate is made of glass. In some embodiments, the second binding moieties on the substrate contain one or more streptavidin groups. In some embodiments, the first binding moiety on the hydrogel-binding probe contains a biotin group. In some embodiments, the hydrogel-binding probe binds to the substrate via the biotin-streptavidin interaction. In some embodiments, the analyte is attached to the substrate via the hydrogel-binding probe.

In some embodiments, the method for determining the location of an analyte in a biological sample further includes extending a 3′ end of the hydrogel-binding probe to generate an extended probe using the analyte bound to the first capture domain as a template. In some embodiments, the extended probe is generated using a reverse transcriptase (e.g., any of the reverse transcriptases described herein). In some embodiments, the analyte is removed after extending a 3′ end of the hydrogel-binding probe to generate an extended probe (e.g., cDNA) using the analyte bound to the first capture domain as a template. In some embodiments, the analyte is removed with RNase (e.g., RNase H) digestion after extending a 3′ end of the hydrogel-binding probe to generate an extended probe using the analyte as a template.

In some embodiments, the method for determining the location of an analyte in a biological sample further includes adding a capture sequence to the 3′ end of the extended probe. In some embodiments, the capture sequence is added to the 3′ end of the extended probe using one or more enzymes. In some embodiments, the capture sequence is added to the 3′ end of the extended probe using a terminal deoxynucleotidyl transferase (TdT). In some embodiments, the capture sequence is added to the 3′ end of the extended probe using a DNA polymerase. In some embodiments, the capture sequence is added to the 3′ end of the extended probe using a DNA ligase. In some embodiments, the capture sequence includes a homopolymeric sequence. In some embodiments, the homopolymeric sequence is a poly(A) sequence. In some embodiments, the capture sequence is a poly(dA) oligonucleotide. In some embodiments, the capture sequence can bind to a poly(dT) sequence, such as, for example a poly(dT) capture domain.

In some embodiments, contacting the substrate includes contacting the extended probe (e.g., cDNA) with an array including a plurality of capture probes, where a capture probe includes (i) a second capture domain, where the second capture domain binds the capture sequence on the extended probe, and (ii) a spatial barcode. In some embodiments, an extended probe binds to a corresponding capture probe via the capture sequence on the extended probe and the second capture domain on the capture probe. In some embodiments, the array having a plurality of capture probes can be any array including spatially barcoded capture probes described herein.

In some embodiments, extending the extended probe to generate a first strand using the capture probe as an extension template. In some embodiments, the extended probe on the substrate is extended using a DNA polymerase using the capture probe as an extension template. In some embodiments, the 3′ end of the capture probe on the array is blocked while the extended probe on the substrate is extended. In some embodiments, the blocking probe can have a 3′ overhang (e.g., poly(dT) sequence). In some embodiments, blocking all or a portion of the capture probe on the array allows the array to be re-used to determine a location of a second analyte in a second biological sample. In some embodiments, the area of the array is smaller than the area of the substrate. In some embodiments, another array with different spatial barcodes can be used to prime the extended probes on a “shifted position” (e.g., about 100 μm shift) to cover a larger area of the biological sample.

In some embodiments, extending the capture probe on the array to generate an extended capture probe includes using the extended probe on the substrate as an extension template. In some embodiments, the capture probe on the array is extended using a DNA polymerase using the extended probe on the substrate as an extension template.

In some embodiments, the method for determining the location of an analyte in a biological sample further includes determining the sequence of (i) the spatial barcode, or a complement thereof, and (ii) all or a portion of a sequence corresponding to the analyte, or a complement thereof, and using the sequences of (i) and (ii) to determine the location of the analyte in the biological sample. In some embodiments, determining the sequence includes sequencing the analyte (e.g., sequencing by any of the methods described herein).

### Compositions

The present disclosure also features compositions for capturing analytes from an embedded biological sample and spatially barcoding analytes with an array.

Thus, provided herein are compositions including a hydrogel-binding probe that includes (i) a first capture domain, where the first capture domain is hybridized to an analyte (e.g., a nucleic acid); (ii) a first binding moiety; and (iii) a hydrogel-binding moiety.

In some embodiments, the first capture domain includes a poly(T) sequence. In some embodiments, the hydrogel-binding probe includes a cleavage domain, a unique molecular identifier, one or more functional domains, or any combination thereof.

In some embodiments, the first binding moiety includes streptavidin. In some embodiments, the first binding moiety includes biotin. In some embodiments, the analyte is a nucleic acid. In some embodiments, the nucleic acid is RNA. In some embodiments, the RNA is mRNA. In some embodiments, the first binding moiety and the second binding moiety include the use of the SpyTag/SpyCatcher system.

In some embodiments, the hydrogel-binding moiety includes an acrydite moiety. In some embodiments, the hydrogel-binding moiety (e.g., acrydite) binds a hydrogel subunit. In some embodiments, the hydrogel subunits includes acrylamide. In some embodiments, the hydrogel subunits form a hydrogel (e.g., a hydrogel embedded in the biological sample). In some embodiments, the hydrogel is a polyacrylamide gel. In some embodiments, the biological sample is removed (e.g., degraded with one or more permeabilization reagents, proteinases, lipases, DNases, RNases etc.).

Also provided herein are compositions including a hydrogel-binding probe that includes (i) a first capture domain, where the first capture domain is hybridized to an analyte (e.g., nucleic acid); (ii) a first binding moiety; and (iii) a hydrogel-binding moiety; where the first binding moiety is bound to a second binding moiety on a substrate.

In some embodiments, the first binding moiety is streptavidin and the second binding moiety is biotin. In some embodiments, the first binding moiety is biotin and the second binding moiety is streptavidin. In some embodiments, the first binding moiety and the second binding moiety include the use of the SpyTag/SpyCatcher system.

In some embodiments, the hydrogel-binding probe includes a cleavage domain, a unique molecular identifier, one or more functional domains, or any combination thereof.

In some embodiments, the hydrogel-binding moiety includes an acrydite moiety. In some embodiments, the hydrogel-binding moiety (e.g., acrydite) binds a hydrogel subunit. In some embodiments, the hydrogel subunits includes acrylamide. In some embodiments, the hydrogel subunits form a hydrogel (e.g., a hydrogel embedded in the biological sample). In some embodiments, the hydrogel is a polyacrylamide gel. In some embodiments, the biological sample is removed (e.g., degraded with one or more permeabilization reagents, proteinases, lipases, DNases, RNases etc.).

In some embodiments, the first capture domain includes a poly(T) sequence. In some embodiments, the analyte is a nucleic acid. In some embodiments, the nucleic acid is mRNA.

In some embodiments, the first capture domain is extended using the analyte (e.g., mRNA) as a template to generate an extended probe. In some embodiments, the first capture domain is extended by a reverse transcriptase. In some embodiments, a capture sequence is added to the extended probe. In some embodiments, one or more polynucleotides are added to the extended probe. In some embodiments, the one or more polynucleotides added to the extended probe (e.g., a capture sequence) include a heteropolymeric sequence. In some embodiments, the one or more polynucleotides added to the extended probe (e.g., a capture sequence) include a homopolymeric sequence. In some embodiments, the homopolymeric sequence is a poly(A) sequence. In some embodiments, the homopolymeric sequence is added by terminal deoxynucleotidyl transferase. In some embodiments, the homopolymeric sequence is added by a poly(A) polymerase. In some embodiments, the homopolymeric sequence is ligated to the extended probe by a ligase.

In some embodiments, after extension of the extended probe (e.g., by a reverse transcriptase), the analyte (e.g., nucleic acid (e.g., mRNA)) is removed from the extended probe. In some embodiments, the analyte is removed by denaturation. In some embodiments, denaturation includes the use of heat. In some embodiments, denaturation includes the use of KOH. In some embodiments, the analyte is removed with an RNase (e.g., RNase H). In some embodiments, the capture sequence added to the extended probe can be added after extension but before removal of the analyte. In some embodiments, the capture sequence added to the extended probe can be added after extension and after removal of the analyte.

Also provided herein are compositions including: (a) an extended probe (e.g., as described herein) including a capture sequence bound on a substrate (e.g., bound by the interaction between a first binding moiety and a second binding moiety) and (b) a second substrate including a plurality of capture probes, wherein a capture probe of the plurality of capture probes includes a spatial barcode and a second capture domain, where the substrate and the second substrate are aligned such that the capture sequence of the extended probe binds to the second capture domain of the capture probe.

In some embodiments, the first binding moiety is streptavidin and the second binding moiety is biotin. In some embodiments, the first binding moiety is biotin and the second binding moiety is streptavidin. In some embodiments, the first binding moiety and the second binding moiety include the use of the SpyTag/SpyCatcher system.

In some embodiments, the second capture domain is a poly(T) sequence. In some embodiments, the capture probe includes a cleavage domain, a unique molecular identifier, one or more functional domains, or any combination thereof.

In some embodiments, the extended probe is further extended using the capture probe as a template. For example, the extended probe can be extended with a polymerase such that the domains (e.g., one or more functional domains, unique molecular identifier, and/or the spatial barcode) of the capture probe (e.g., complements thereof) are incorporated into the extended probe, thereby spatially barcoding the analyte (e.g., nucleic acid).

In some embodiments, the capture probe is extended using the extended probe as a template. For example the capture probe can be extended with a polymerase such that complement of the extended probe (e.g., the proxy of the captured analyte) is incorporated into the capture probe, thereby spatially barcoding the analyte (e.g., nucleic acid). In some embodiments, the further extended probe is released from the capture probe to which it is hybridized. In some embodiments, the releasing is by denaturation. In some embodiments, denaturation includes the use of heat. In some embodiments, denaturation includes the use of KOH.

In some embodiments, the 3′ end of the capture probe is blocked (e.g., blocked by any of the methods described herein). In some embodiments, blocking the 3′ end of the capture probe only allows extension of the extended probe where the extended probe incorporates the domains of the capture probe. Extension of the capture probe (e.g., extension of the 3′ end) does not occur when the 3′ end of the capture probe (e.g., the capture domain of the capture probe) is blocked. In such embodiments, the array including the plurality of capture probes can be re-used (e.g., contacted) with another substrate where analytes (e.g., nucleic acids) have been captured from an embedded biological sample and bound to substrate via first and second binding moieties or a biological sample.

### Kits

In addition to the methods and compositions provided herein, the present disclosure also features kits for capturing analytes from an embedded biological sample and spatially barcoding the analytes with an array.

Provided herein are kits including (a) a plurality of hydrogel-binding probes that include (i) a first capture domain; (ii) a first binding moiety; and (iii) a hydrogel-binding moiety, (b) a substrate including a plurality of second binding moieties, (c) an array including a plurality of capture probes, where a capture probe of the plurality of capture probes includes a spatial barcode and a second capture domain, and (d) a plurality of hydrogel subunits (e.g., any of the hydrogel subunits described herein).

In some embodiments, the kit includes one or more permeabilization reagents (e.g., proteases, lipases, DNase, RNases, detergents, and combinations thereof). In some embodiments, the first binding moiety is biotin and the second binding moiety is streptavidin. In some embodiments, the first binding moiety is streptavidin and the second binding moiety is biotin. In some embodiments, the first binding moiety and the second binding moiety include the use of the SpyTag/SpyCatcher system.

In some embodiments, the first capture domain and/or the second capture domain includes a poly(T) sequence.

In some embodiments, the hydrogel-binding moiety is acrydite. In some embodiments, the hydrogel-binding moiety (e.g., acrydite) interacts with one or more hydrogel subunits. In some embodiments, the plurality of hydrogel subunits include acrylamide.

In some embodiments, the kit includes a terminal deoxynucleotidyl transferase, a ligase, and/or a poly(A) polymerase. For example, the terminal deoxynucleotidyl transferase and/or the poly(A) polymerase can add one or more additional nucleotides to an extended probe (e.g., adding a capture sequence as described herein). In some embodiments, the ligase can ligate a poly(A) sequence (e.g., a capture sequence) to the extended probe.

In some embodiments, the kit includes a reverse transcriptase. In some embodiments, the kit includes a polymerase (e.g., a DNA polymerase). In some embodiments, the kit includes instructions for performing any of the methods described herein.

### EXAMPLES

### Example 1. Analyte Capture from an Embedded Biological Sample

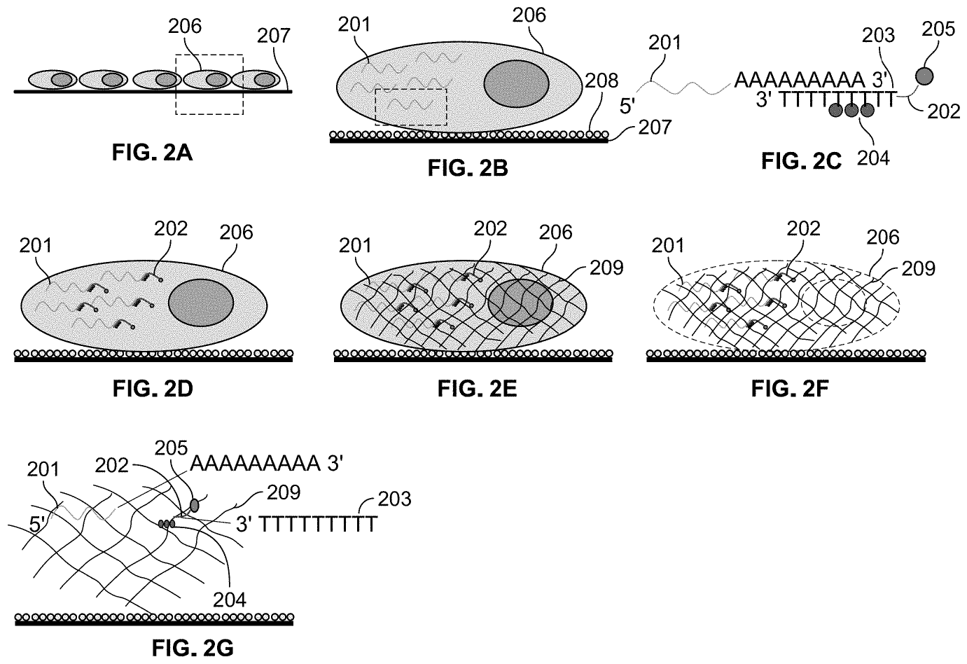

FIGS. 2A-2G and 3A-31I show an exemplary method for determining the location of an analyte in a biological sample with a reusable spatial array. FIG. 2A shows a biological sample 206 on a substrate 207. In some embodiments, a biological sample is placed on a streptavidin-coated substrate 207 (e.g., a glass slide). In some embodiments, the biological sample adheres to the streptavidin-coated substrate 207 (e.g., slide). In some embodiments, the biological sample is stained (e.g., H&E, DAPI, etc.), imaged and/or permeabilized. FIG. 2B shows a biological sample 206 located near a substrate 207. In some embodiments, the biological sample 206 includes one or more analytes 201. In some embodiments, the analyte 201 is an mRNA. In some embodiments, the substrate 207 includes one or more second binding moieties 208. In some embodiments, the second binding moiety 208 is a streptavidin group. FIG. 2C shows an exemplary scheme of an analyte 201 bound to a hydrogel-binding probe 202. In some embodiments, the analyte 201 is mRNA. In some embodiments, the hydrogel-binding probe 202 contains a first capture domain 203, one or more first binding moieties 204, and a hydrogel-binding moiety 205. In some embodiments, the first capture domain 203 is a poly(dT) nucleotide. In some embodiments, the first binding moieties 204 are biotin groups. In some embodiments, the hydrogel-binding moiety 205 has an acrydite moiety. In some embodiments, the mRNA 201 inside the biological sample are hybridized in situ with hydrogel-binding probes 202.

FIG. 2D shows a hydrogel binding probe 202 binding to an analyte 201 when the biological sample 206 is contacted with a plurality of hydrogel binding probes 202. FIG. 2E shows that the hydrogel-binding probes 202 are cross-linked to a hydrogel 209 via the hydrogel-binding moiety 205 when the biological sample 206 is embedded with a hydrogel 209 that contains a plurality of hydrogel subunits. In some embodiments, one or more hydrogel subunits include acrylamide. In some embodiments, the hydrogel 209 is a polyacrylamide hydrogel. In some embodiments, the biological sample 206 is perfused with an acrylamide solution which forms a mesh which binds to the acrydite moiety 205 of the hydrogel-binding probe 202. In some embodiments, the analyte 201 is captured by the hydrogel 209 via the hydrogel-binding probe 202. FIG. 2F shows that the remainder of the biological sample 206 is cleared (e.g., disrupted or dissolved and removed) after with the analytes 201 are captured by the hydrogel 209. In some embodiments, clearing the biological sample 206 includes the use of one or more of a proteases, a lipase, an RNAse and a DNAse. In some embodiments, the biological sample 206 is cleared with Proteinase K digestion. In some embodiments, the one or more second binding moieties 208 on the substrate 207 and the one or more first binding moieties 204 on the hydrogel-binding probe 202 are resistant to Proteinase K. In some embodiments, the second binding moieties 208 are proteinase K resistant streptavidin (see, e.g., Hytonen, V. P., et al., Design and Construction of Highly Stable Protease-resistant Chimeric Avidins, The Journal of Biological Chemistry, 280, 10228-1033 (2005)). In some embodiments, clearing the biological sample includes the use of one or more washes. In some embodiments, after Proteinase K treatment, peptides, lipids, and DNA are removed by one or more washes and only the cross-linked analytes (e.g., mRNA) 201 are left in the acrylamide mesh 209.

FIG. 2G is an exemplary scheme showing an analyte 201 bound to a hydrogel-binding probe 202 which is in turn bound to the hydrogel 209. In some embodiments, the hydrogel 209 is a polyacrylamide hydrogel. In some embodiments, the analyte 201 is mRNA. In some embodiments, the hydrogel-binding probe 202 contains a first capture domain 203, one or more first binding moieties 204, and a hydrogel-binding moiety 205. In some embodiments, the first capture domain 203 is a poly(T) capture domain. In some embodiments, the one or more first binding moieties 204 are biotin groups. In some embodiments, the hydrogel-binding moiety 205 has an acrydite moiety. In some embodiments, the captured mRNA analyte 201 is released from the mesh 209. In some embodiments, the analyte 201 is released from the hydrogel 209 by disassembling the hydrogel 209. In some embodiments, the hydrogel 209 has a reversible cross-linker. In some embodiments, the analyte 201 is released from the hydrogel 209 by disassembling the hydrogel-binding probe 202. In some embodiments, the hydrogel-binding probe 202 has a cleavage domain, a unique molecular identifier, one or more functional domains, or combinations thereof. In some embodiments, the analyte 201 is released from the hydrogel 209 by enzymatic cleavage of the hydrogel-binding probe 202. After releasing the analyte 201 bound to the hydrogel-binding probe from the hydrogel, the analyte 201 bound to the hydrogel-binding probe is migrated to the substrate 207. In some embodiments, the analyte 201 is migrated to the substrate 207 by active migration. In some embodiments, the analyte 201 is migrated to the substrate 207 by electrophoresis. In some embodiments, the analyte 201 bears a negative or positive charge. In some embodiments, the hydrogel-binding probe 202 bears a negative or positive charge. In some embodiments, the hydrogel-binding probe 202 includes a magnetic particle. In some embodiments, the analyte 201 is migrated to the substrate 207 by a magnetic force. In some embodiments, the substrate 207 is made of glass. In some embodiments, the second binding moieties 208 on the substrate 207 include one or more streptavidin groups. In some embodiments, the first binding moiety 204 on the hydrogel-binding probe 202 contains a biotin group. In some embodiments, the hydrogel-binding probe 202 binds to the substrate 207 via biotin-streptavidin interaction. In some embodiments, the analyte 201 is attached to the substrate 207 via the hydrogel-binding probe 202.

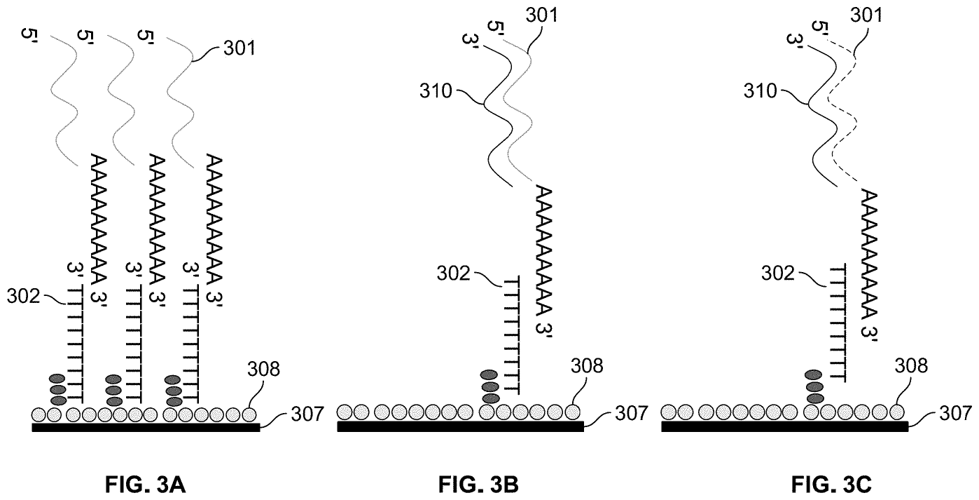

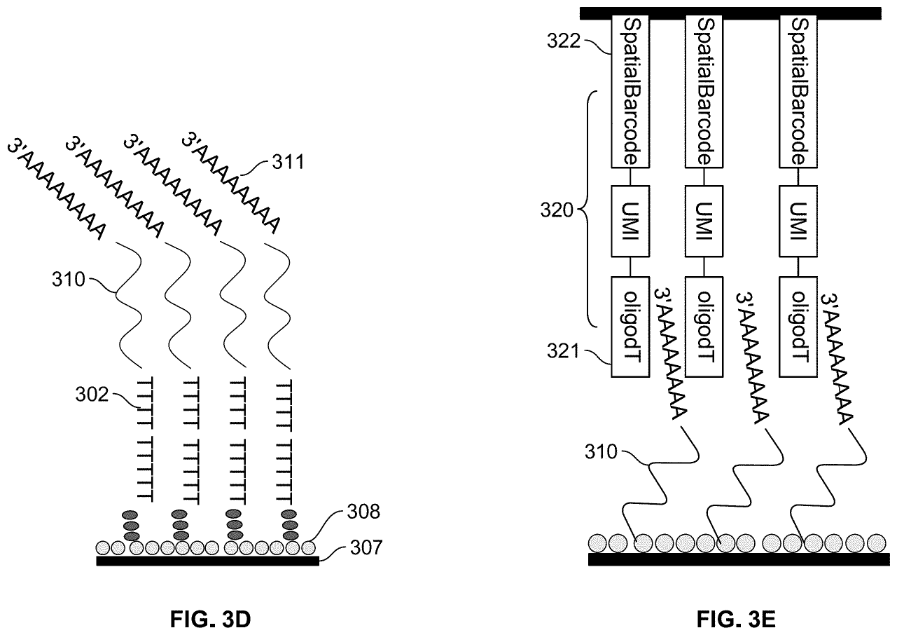

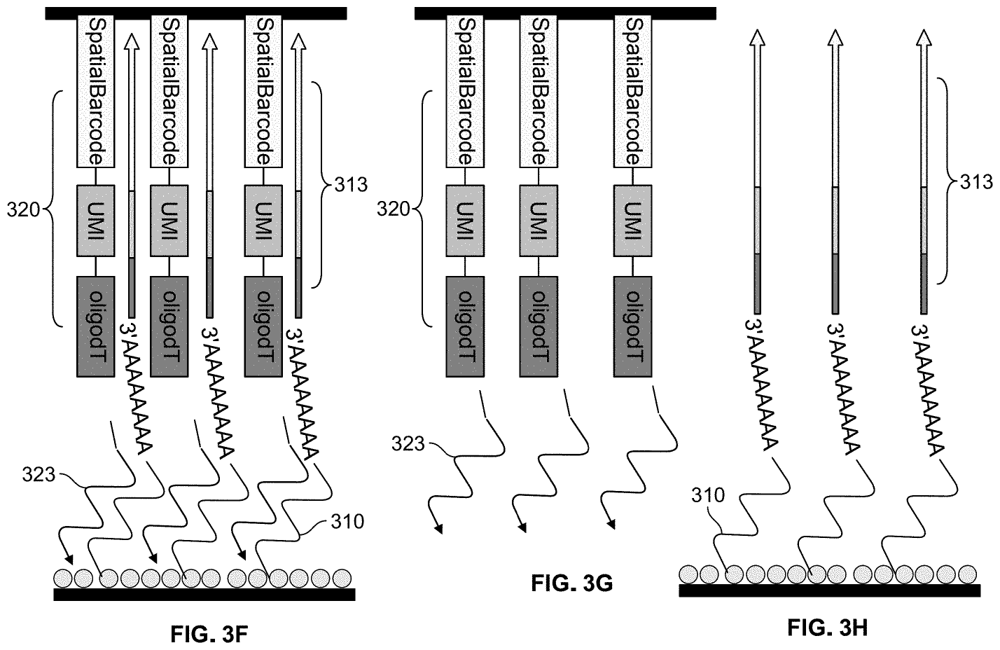

FIG. 3A shows that the analyte 301 is attached to the substrate 307 via the hydrogel-binding probe 302. In some embodiments, the hydrogel-binding probes 302 bind to the glass surface 307 via biotin-streptavidin interaction and the analyte 301 stays bound to the hydrogel-binding probe 302.

FIG. 3B shows that the 3′ end of the hydrogel-binding probe 302 is used to prime an extension reaction using the analyte 301 as a template 301 to generate an extended probe 310. In some embodiments, the analyte 301 is mRNA. In some embodiments, the extended probe 310 is cDNA. In some embodiments, the 3′ end of the hydrogel-binding probe 302 is extended using a reverse transcriptase (e.g., any of the reverse transcriptases described herein) using the analyte 301 as a template for an extension reaction.

FIG. 3C shows that the analyte 301 is removed after an extended probe 310 is generated using the analyte 301 as an extension template. In some embodiments, the analyte 301 is mRNA. In some embodiments, the analyte 301 is removed using RNaseH digestion. In some embodiments, the extended probe (e.g., single-stranded cDNA) 310 is still linked to the substrate 307 after the removal of analyte 301.

FIG. 3D shows that a capture sequence 311 is added to the 3′ end of the extended probe 310. In some embodiments, the capture sequence 311 includes a homopolymeric sequence. In some embodiments, the homopolymeric sequence is a poly(A) sequence. In some embodiments, the capture sequence is a poly(A) oligonucleotide. In some embodiments, the capture sequence 311 is added using one or more enzymes. In some embodiments, the capture sequence 311 is added using a terminal deoxynucleotidyl transferase. In some embodiments, the capture 311 sequence is added to the 3′ end of the extended probe 310 using a poly(A) polymerase. In some embodiments, the capture sequence 311 is added to the 3′ end of the extended probe 310 using a DNA ligase (e.g., a poly(A) oligonucleotide ligation reaction).

FIG. 3E shows that the extended probes 310 are contacted with an array having a plurality of capture probes 320, wherein a capture probe 320 has (i) a second capture domain 321 (e.g., a poly(dT) sequence), wherein the second capture domain 321 binds the capture sequence 311 of an extended probe 310, and (ii) a spatial barcode 322. The array having a plurality of capture probes 320 can be any array including spatially barcoded capture probes described herein. In some embodiments, a capture probe 320 contains (i) a second capture domain 321, (ii) a spatial barcode 322, and (iii) a unique molecular identifier (UMI). In some embodiments, the second capture domain 321 is a poly(dT) sequence capable of capturing mRNA. In some embodiments, an extended probe 310 binds to a capture probe 320 via binding between a second capture domain 321 and capture sequence 311.

FIG. 3F shows that an extended probe 310 and a capture probe 320 can prime each other to make complementary strands of each other. In some embodiments, the extended probe 310 can act as a primer to generate a first strand 313 using a capture probe 320 as an extension template. In some embodiments, the capture probe 320 can also act as a primer to generate an extended capture probe 323 using an extended probe 310 as an extension template. In some embodiments, the 3′ end of the capture probes 320 can be blocked so that only the extended probe 310 is extended to generate a complement 313. In some embodiments, the array of capture probes 320 can be re-used for another biological sample or another substrate where analytes (e.g., nucleic acids) have been captured from an embedded biological sample and bound to a substrate via a first and second binding moiety. In some embodiments, the first strand 313 and the extended capture probe 323 are generated using a DNA polymerase.

FIG. 3G shows that the capture probe 320 on the array can be used as a primer to generate an extended capture probe 323 using a corresponding extended probe 310 on the substrate 307 as an extension template.

FIG. 311 shows that the extended probe 310 on the substrate 307 can be used as a primer to generate a first strand 313 using a corresponding capture probe 320 on the array as an extension template.

What is claimed is:

1. A method for determining a location of an analyte in a biological sample, the method comprising:
(a) contacting the biological sample with a plurality of hydrogel-binding probes, wherein a hydrogel-binding probe of the plurality of hydrogel-binding probes comprises (i) a first capture domain, wherein the first capture domain binds to the analyte; (ii) a first binding moiety; and (iii) a hydrogel-binding moiety;
(b) embedding the biological sample in a hydrogel comprising a plurality of hydrogel subunits, wherein the hydrogel-binding probe is crosslinked to a hydrogel subunit of the plurality of hydrogel subunits via the hydrogel-binding moiety;
(c) releasing the analyte bound to the hydrogel-binding probe from the hydrogel, thereby allowing the analyte bound to the hydrogel-binding probe to bind to a substrate comprising a plurality of second binding moieties, wherein a second binding moiety of the plurality of second binding moieties binds to the first binding moiety;
(d) extending a 3′ end of the hydrogel-binding probe to generate an extended probe using the analyte bound to the first capture domain as a template and adding a capture sequence to the 3′ end of the extended probe;
(e) contacting the substrate comprising the extended probe with an array comprising a plurality of capture probes, wherein a capture probe comprises (i) a second capture domain, wherein the second capture domain binds the capture sequence of the extended probe, and (ii) a spatial barcode;
(f) extending the extended probe to generate a first strand using the capture probe as a template; and
(g) determining the sequence of (i) the spatial barcode, or a complement thereof, and (ii) all or a portion of a sequence corresponding to the analyte, or a complement thereof, and using the sequences of (i) and (ii) to determine the location of the analyte in the biological sample.

2. The method of claim 1, wherein step (f) further comprises extending the capture probe on the array to generate a second strand using the extended probe as a template.

3. The method of claim 1, further comprising, before step (f), blocking the 3′ end of the extended capture probe on the array.

4. The method of claim 3, wherein the hydrogel-binding probe further comprises a cleavage domain, a unique molecular identifier, one or more functional domains, or any combination thereof.

5. The method of claim 1, wherein the analyte is RNA, and optionally, wherein the RNA is mRNA.

6. The method of claim 1, wherein the first capture domain of the hydrogel-binding probe is a poly(T) sequence, the second capture domain comprises a poly(T) sequence, and the capture sequence comprises a homopolymeric sequence, wherein the homopolymeric sequence is a poly(A) sequence.

7. The method of claim 1, wherein the hydrogel-binding moiety is located at a 5′ end of the hydrogel-binding probe and wherein the hydrogel-binding moiety comprises acrydite.

8. The method of claim 1, wherein the method further comprises, after step (b), and before step (c) clearing the biological sample, wherein the clearing comprises the use of one or more of a lipase, an RNase, a DNase, and a protease, wherein the protease comprises Proteinase K and, optionally, one or more washing steps.

9. The method of claim 8, wherein the plurality of second binding moieties and the first binding moiety of the hydrogel-binding probe are resistant to degradation by proteinase K.

10. The method of claim 1, further comprising, after step (d), removing the analyte from the hydrogel, wherein the removing comprises the use of an RNase.

11. The method of claim 1, wherein the method further comprises, before step (a), a step of permeabilizing the biological sample and optionally before step (b) staining and imaging the biological sample.

12. The method of claim 1, wherein step (c) comprises disassembling the hydrogel, wherein the hydrogel comprises a reversible cross-linker.

13. The method of claim 1, wherein step (c) comprises actively migrating the analyte bound to the hydrogel-binding probe to the substrate, wherein the step of actively migrating the analyte bound to the hydrogel-binding probe to the substrate comprises electrophoresis.

14. The method of claim 1, wherein the hydrogel-binding probe further comprises an agent that decreases migration of the analyte bound to the hydrogel-binding probe.

15. The method of claim 14, wherein step (c) further comprises removing the agent that decreases migration from the hydrogel-binding probe and after removing the agent that decreases migration from the hydrogel-binding probe, a step of actively migrating the analyte bound to the hydrogel-binding probe to the substrate via electrophoresis.

16. The method of claim 1, wherein the hydrogel-binding probe comprises a magnetic particle and wherein step (c) comprises actively migrating the analyte bound to the hydrogel-binding probe to the substrate using a magnetic field.

17. The method of claim 1, wherein the hydrogel-binding probe comprises a charged particle and wherein step (c) comprises actively migrating the analyte bound to the hydrogel-binding probe to the substrate using electrophoresis.

18. The method of claim 1, wherein the adding of the capture sequence to the 3′ end of the extended probe in step (d) comprises the use of one or more enzyme(s), wherein the one or more enzymes is a DNA ligase, a terminal deoxynucleotidyl transferase, or a poly(A) polymerase.

19. The method of claim 1, wherein the extending of the 3′ end of the hydrogel-binding probe in step (d) is performed using a reverse transcriptase and wherein the extending of the 3′ end of the extended probe in step (f) is performed using a DNA polymerase.

20. The method of claim 1, wherein the first binding moiety comprises biotin and the second binding moiety comprises streptavidin or the first binding moiety comprises streptavidin and the second binding moiety comprises biotin, the plurality of hydrogel subunits comprise acrylamide, and the hydrogel comprises a polyacrylamide hydrogel.
